(19) United States
(12) Patent Application Publication
(10) Pub. No.: US 2024/0285716 A1
(43) Pub. Date: Aug. 29, 2024

(54) PROCESS FOR PURIFICATION OF MITRAGYNINE FROM CRUDE EXTRACT

(71) Applicant: LUCKY 4U EXIMS, Tirupati (IN)

(72) Inventors: Surya Pavan Kumar Avva, Tirupati (IN); V.S.R. Suresh Mamidipalli, Tirupati (IN)

(21) Appl. No.: 18/311,722

(22) Filed: May 3, 2023

(30) Foreign Application Priority Data
Feb. 17, 2023 (IN) 202341010938

(51) Int. Cl. A61K 36/74; A61K 31/4375

(52) U.S. Cl. CPC A61K 36/74; A61K 31/4375; A61K 2236/13; A61K 2236/17; A61K 2236/331; A61K 2236/35; A61K 2236/39; A61K 2236/55

(57) ABSTRACT

The present invention discloses a process for purification of Mitragynine or preparation of purified Mitragynine salt from crude Mitragynine extract for the pharmaceutical preparations. More particularly, the present invention discloses purification of Mitragynine or preparation of purified Mitragynine salt using polar aprotic solvents.

### CROSS-REFERENCE TO RELATED APPLICATION

This application claims foreign priority benefits under 35 U.S.C. § 119 from Indian patent application Ser. No. 202341010938, filed Feb. 17, 2023, the content of which is hereby incorporated by reference in its entirety.

### TECHNICAL FIELD

The present invention relates to a process for purification of alkaloids, more specifically a process for purification of Mitragynine alkaloid. Particularly present invention relates to purification of Mitragynine or preparation of purified Mitragynine salt from crude Mitragynine extract for the pharmaceutical preparations. More particularly, present invention relates to purification of Mitragynine or preparation of purified Mitragynine salt using polar aprotic solvents.

### BACKGROUND

Alkaloids are a group of naturally occurring chemical compounds that mostly contain basic nitrogen atoms. They are of plant origin and have pronounced physiological actions on humans. Alkaloids occur in plants usually in mixtures of related compounds together with inert constituents, such as tannins, proteins, fats, resins, and pigments, which generally hinder their isolation.

Mitragynine is an alkaloid and the most abundant active alkaloid in the Southeast Asian plant Mitragyna speciosa, commonly known as kratom. It is found in the leaves of tree Mitragyna speciosa. Of all the other alkaloids present in plant Mitragyna speciosa, Mitragynine is considered to be largely responsible for its therapeutic activity.

Mitragynine was first isolated in 1921 and the chemical structure of Mitragynine was fully elucidated in 1964. The systematic (IUPAC) name of Mitragynine is methyl (E)-2-[(2S,3S,12bS)-3-ethyl-8-methoxy-1,2,3,4,6,7,12,12b-octahydroindolo[2,3-a]quinolizin-2-yl]-3-methoxyprop-2-enoate. The chemical structure of Mitragynine is represented as:

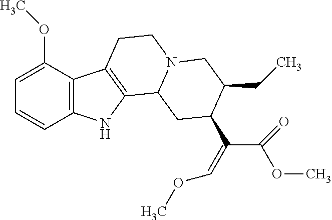 [SMILES: CC[C@@H]1CN2CCc3c([nH]c4cccc(OC)c34)C2C[C@@H]1/C(=C\OC)C(=O)OC]

Mitragynine possesses several pharmacological properties such as analgesic, anti-inflammatory, antipyretic, antidiarrheal, euphoric, antidepressant, and anxiolytic effects.

There are many processes known for extraction of Mitragynine from the leaves of Mitragyna Speciosa plant. The obtained Mitragynine extract includes many impurities and hence purification plays an important role after extraction of Mitragynine from the plant. Some of the methods know in the art are:

Radhiahtul Raehan Mustafa et. al., Enhancing extraction yield and purity of Mitragynine from Mitragyna speciosa through sequential solvent extraction and characterisation using NMR technique. Int J Sci Technol Res 9(3): 3846-54, March 2020, discloses separation of Mitragynine from the crude hexane-chloroform-MeOH extract using silica gel column chromatography. The solvent system used in column chromatography is hexane: EtOAc. Approximately 1.0 g of the crude extract was purified using column chromatography.

Laura Orio et. al., UAE, MAE, SFE-CO2 and classical methods for the extraction of Mitragyna speciosa leaves, Ultrasonics Sonochemistry 19, 591-595, 2012, discloses Mitragynine purification method using flash chromatography using petroleum ether and ethyl acetate as eluent, on a 4 g silica gel column with a flow rate of 18 ml/min and detected at UV 254 nm. The fraction containing the Mitragynine was collected, and the solvent was removed using rotary evaporator.

Neni Isnaeni et. al., Preliminary Study of Isolation and Purification Mitragynine from Kratom Leaves, Materials Science Forum (Volume 1061), 173-179, May 2022 discloses purification of Mitragynine extract by column chromatography, eluted with a mixture of n-hexane-ethyl acetate-25% ammonia with a ratio of 30:15:1 (v/v).

Luisa Boffa et. al., Alkaloid Profiles and Activity in Different Mitragyna speciosa Strains, Natural Product Communications Vol. 13 (9) 2018, discloses extraction and purification of various alkaloids from different Mitragyna speciosa strains which includes extraction of M. speciosa leaves with a mixture of methanol and water with the help of a magnetic stirrer at room temperature for 24 hours. This is followed by purification including several steps using either an alkaline phase or an acid phase ending with precipitation at pH 9 (ammonium hydroxide solution). The residue and alkaline solution were rinsed with Dichloromethane.

The available processes for purification of Mitragynine have drawbacks such as these processes have many steps and are time consuming or involves use of expensive instruments. This results in potential sample losses, poor accuracy and high cost.

By aforementioned facts, there exists a need for development of simple and commercially significant process for purification of Mitragynine.

### SUMMARY

The primary object of the present invention is to provide a process for purification of Mitragynine or preparation of purified Mitragynine salt from crude Mitragynine extract.

Another object of the present invention is to provide a process for purification of Mitragynine or the preparation of purified Mitragynine salt using polar aprotic solvents.

Another object of the present invention is to provide a facile and efficient process for purification of an extracted Mitragynine which results in an optimum purity of Mitragynine salt.

Further object of the present invention is to provide a high purity Mitragynine oxalate salt for pharmaceutical applications.

Another object of the invention is to provide a process resulting in improved yield of Mitragynine oxalate salt (32%) with purity of 62-68% by HPLC.

Yet another object of the present invention is to provide an improved process for purification of an extracted Mitragynine which is cost effective and feasible for industrial scale production.

Accordingly, in one aspect the present invention provides a process for the purification of Mitragynine from the crude extract, comprising the steps of:
(a) mixing crude extract of Mitragynine with polar aprotic solvent in a reactor followed by stirring;
(b) filtering the stirred solution obtained in step (a) to obtain filtered solution and spent;
(c) optionally, adding polar aprotic solvent to the spent obtained in step (b), followed by stirring and filtering, to obtain filtered solution;
(d) mixing the filtered solution obtained in step (b) and/or step (c) and an acid solution in the reactor followed by stirring until Mitragynine salt precipitate is formed;
(e) filtering the precipitated salt as obtained in step (d);
(f) washing the filtered precipitated salt of step (e) with polar aprotic solvent to obtain the salt product;
(g) optionally, performing step (f) multiple times to obtain the salt product; and
(h) drying the salt product obtained in step (f) and/or step (g) to form purified Mitragynine salt.

The polar aprotic solvent employed in steps (a) and (c) of the present process is selected from a group comprising of acetone, acetonitrile, dimethyl sulfoxide, dimethylformamide, tetrahydrofuran, dichloromethane, ethyl acetate, or mixture thereof, preferably the solvent is acetone.

In step (a) of the present process, the stirring is carried out for 2 hours at 30-50 RPM.

In step (b) of the present process, filtering is carried out through cleaned nutsche filter on 5 microns cloth.

In the present process, the acid solution in step (d) is an organic or mineral acid solution.

The organic or mineral acid solution of the present process is prepared by dissolving the organic or mineral acid slowly into the solvent with continuous mixing followed by stirring at 30-50 RPM for 1 hour and filtering through 100 mesh cloth.

The organic or mineral acid is selected from a group comprising of hydrochloric acid, hydrobromic acid, perchloric acid, sulphuric acid, oxalic acid, tartaric acid, citric acid, formic acid, trifluoro acetic acid, trichloro acetic acid, or mixture thereof, preferably the organic is oxalic acid.

The solvent used in preparation of organic or mineral acid solution is selected from a group comprising of chloroform, acetone, methanol, ethanol, n-butanol, isopropyl alcohol, ethyl acetate, diethyl ether, n-hexane, methylene dichloride, ethylene dichloride or mixture thereof, preferably the solvent is acetone.

The organic acid solution of the present process is oxalic acid solution.

In step (d) of the present process, the stirring is carried out for 2 hours at 30-50 RPM.

The Mitragynine salt precipitate formed in step (d) of the present process is hydrochloride salt, hydrobromide salt, perchlorate salt, sulphate salt, oxalate salt, tartrate salt, citrate salt, formate salt, trifluoro acetate salt, or trichloro acetate salt.

Preferably the Mitragynine salt precipitate formed in step (d) of the present process is Mitragynine oxalate salt precipitate.

The polar aprotic solvent employed in step (f) of the present process is selected from a group comprising of acetone, acetonitrile, dimethyl sulfoxide, dimethylformamide, tetrahydrofuran, dichloromethane, ethyl acetate, or mixture thereof, preferably the solvent is acetone.

In step (h) of the present process, the salt product is air dried for 5-6 hours followed by drying in Vacuum Tray Dryer at 45-50° C. under 700 mm of Hg.

The purified Mitragynine salt obtained in step (h) of the present process is purified Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt, preferably the purified Mitragynine salt is purified Mitragynine oxalate salt.

In some embodiment of the invention, there is provided a process for the purification of Mitragynine from the crude extract, said process comprises the steps of:
(a) mixing crude extract of Mitragynine with fresh and purified acetone in the reactor followed by stirring for 2 hours at 30-50 RPM;
(b) filtering the stirred solution as obtained in step (a) to obtain filtered solution and spent;
(c) again adding fresh and purified acetone to the spent obtained in step (b), followed by stirring for 2 hours at 30-50 RPM and filtering, to obtain filtered solution;
(d) mixing the filtered solution obtained in step (b) and step (c) and oxalic acid solution in the reactor followed by stirring at 30-50 RPM for 2 hours until Mitragynine oxalate salt precipitate is formed;
(e) filtering the precipitated Mitragynine oxalate salt as obtained in step (d);
(f) washing the filtered precipitated Mitragynine oxalate salt of step (e) with fresh and purified acetone to obtain the Mitragynine oxalate salt product;
(g) optionally performing step (f) again to obtain the Mitragynine oxalate salt product; and
(h) air drying the salt product obtained in step (f) and/or step (g) for 5-6 hours followed by drying in Vacuum Tray Dryer at 45-50° C. under 700 mm of Hg to form purified Mitragynine oxalate salt.

The oxalic acid solution of step (d) of present process is prepared by dissolving oxalic acid in acetone with continuous mixing followed by stirring for 1 hour at 10-50 RPM and filtering through 100 mesh cloth.

The obtained Mitragynine oxalate salt in the present process is in amorphous form.

The instant process provides improved yield of Mitragynine oxalate salt (32%) with purity of 62-68% by HPLC.

### BRIEF DESCRIPTION OF THE DRAWINGS

FIG. 1 depicts an HPLC graph showing purity of obtained Mitragynine salt.

FIG. 2 depicts an HPLC graph showing purity of obtained Mitragynine salt.

FIG. 3 depicts an HPLC graph showing purity of obtained Mitragynine salt.

### DETAILED DESCRIPTION

The following detailed description refers to the accompanying drawing that show, by way of illustration, specific details and embodiments in which the invention may be practised. These embodiments are described in sufficient detail to enable those skilled in the art to practise the invention. Other embodiments may be utilized, and changes may be made without departing from the scope of the invention. The various embodiments are not necessarily mutually exclusive, as some embodiments can be combined with one or more other embodiments to form new embodiments.

Unless defined otherwise, all technical and scientific terms used herein have the same meaning as commonly understood by one of ordinary skill in the art to which the methods belong.

Further the embodiments described herein can be understood more readily by reference to the following detailed description, examples, and drawings. Methods described herein are merely illustrative of the principles of the present invention and are not limited to the specific embodiments presented in the detailed description, examples, and drawings. Numerous modifications and adaptations will be readily apparent to those of skill in the art without departing from the spirit and scope of the invention.

Where a range of values is provided, it is understood that each intervening value between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

Accordingly, the present invention discloses and describes an improved process for the purification of Mitragynine or preparation of purified Mitragynine salt from the crude extract wherein the process is performed in a reactor, comprising the steps by the process of the invention as described herein.

The present process for purification of Mitragynine comprises first dissolving Mitragynine from crude Mitragynine extract in the solvent followed by adding acid in it to form precipitated salt, which is further washed with solvent to obtain purified Mitragynine salt. The present process provides improved yield of Mitragynine oxalate salt (32%) with purity of 62-68% by HPLC.

In one aspect of the invention, there is provided a process for the purification of Mitragynine from the crude extract comprising the steps of:
(a) mixing crude extract of Mitragynine with polar aprotic solvent in a reactor followed by stirring;
(b) filtering the stirred solution obtained in step (a) to obtain filtered solution and spent;
(c) optionally, adding polar aprotic solvent to the spent obtained in step (b), followed by stirring and filtering, to obtain filtered solution;
(d) mixing the filtered solution obtained in step (b) and/or step (c) and an acid solution in the reactor followed by stirring until Mitragynine salt precipitate is formed;
(e) filtering the precipitated salt as obtained in step (d);
(f) washing the filtered precipitated salt of step (e) with polar aprotic solvent to obtain the salt product;
(g) optionally, performing step (f) multiple times to obtain the salt product; and
(h) drying the salt product obtained in step (f) and/or step (g) to form purified Mitragynine salt.

The crude extract of Mitragynine used in the present invention may be prepared by the methods known in the state of art. Traditionally known methods used for extraction of Mitragynine from plants includes maceration, percolation, reflux, Solvent Extraction, Soxhlet extraction. Amrianto et. al., Mitragynine: a review of its extraction, identification, and purification methods, Current Research on Biosciences and Biotechnology 3(1) 165-171, 2021, discloses various methods reported in the art for extracting Mitragynine, both conventional and renewable technology. Various extraction methods of Mitragynine reported in this literature are Solvent Extraction, Ultrasound assisted extraction (UAE), Accelerated solvent extraction (ASE) etc. The prepared crude extract of Mitragynine is purified by the process provided by the present invention.

### Step (a):

In one embodiment of the present invention, crude extract of Mitragynine is added slowly to the fresh and purified polar aprotic solvent with continuous mixing followed by stirring.

The polar aprotic solvent employed in step (a) may be selected from a group comprising of acetone, acetonitrile, dimethyl sulfoxide, dimethylformamide, tetrahydrofuran, dichloromethane, ethyl acetate, or mixture thereof, preferably the solvent is acetone.

In an embodiment of the present invention, mixing of crude extract with fresh and purified polar aprotic solvent in the reactor according to the step (a) is followed by stirring the reaction the mixture by an agitator at 20-50 RPM, preferably 30-50 RPM, at room temperature for 1-3 hours, most preferably 2 hours.

### Step (b):

In some embodiment of the invention, filtering the stirred solution of step (a) of the above-described process may be done as known in the state of art. In one preferred embodiment the stirred solution of step (a) is filtered through cleaned nutsche filter on 5 microns cloth. The said filtration step results in separation of filtered solution and spent.

### Step (c):

The spent obtained in step (b) of the present process can optionally be treated again with polar aprotic solvent followed by stirring and filtering as defined above.

The polar aprotic solvent employed in step (c) may be selected from a group comprising of acetone, acetonitrile, dimethyl sulfoxide, dimethylformamide, tetrahydrofuran, dichloromethane, ethyl acetate, or mixture thereof, preferably the solvent is acetone.

The stirring as mentioned in the step (c) may be carried out at 20-50 RPM, preferably 30-50 RPM, at room temperature for 1-3 hours, most preferably 2 hours.

In one preferred embodiment the stirred solution is filtered through cleaned nutsche filter on 5 microns cloth.

### Step (d):

In one embodiment, the filtered solution obtained in step (b) is loaded into the reactor. In another embodiment, the filtered solution obtained in step (b) and step (c) are combined and then loaded into the reactor.

In some of the embodiment, the acid solution in step (d) is an organic or mineral acid solution.

In some embodiment of the invention, the organic or mineral acid solution as used in the step (d) of the present invention is prepared by dissolving the organic or mineral acid in the solvent by stirring for 30 minutes-2 hours, preferably 1 hour at 10-50 RPM, followed by filtering. Filtering may be done as known in the state of art or as described above. In one preferred embodiment filtration is carried through 100 mesh cloth.

The organic or mineral acid may be selected from a group comprising of hydrochloric acid, hydrobromic acid, perchloric acid, sulphuric acid, oxalic acid, tartaric acid, citric acid, formic acid, trifluoro acetic acid, trichloro acetic acid, or mixture thereof. In one preferred embodiment of the invention, the acid employed is organic acid, most preferably oxalic acid.

The solvent used in preparation of organic or mineral acid solution may be an organic solvent selected from a group comprising of chloroform, acetone, methanol, ethanol, n-butanol, isopropyl alcohol, ethyl acetate, diethyl ether, n-hexane, methylene dichloride, ethylene dichloride or mixture thereof, preferably the solvent is acetone.

In some embodiment of the invention, step (d) involves mixing of filtered solution obtained in step (b) and/or step (c) and organic or mineral acid solution in the reactor followed by stirring until Mitragynine salt precipitate is formed.

The stirring as mentioned in the step (d) may be carried out in 50 minutes to 3 hours, preferably 2 hours, at 20-50 RPM, preferably 30-50 RPM.

The Mitragynine salt precipitate as formed in step (d) depends upon the organic or mineral acid employed. In one preferred embodiment of the invention, the Mitragynine salt precipitate formed in step (d) of the above-described process is hydrochloride salt, hydrobromide salt, perchlorate salt, sulphate salt, oxalate salt, tartrate salt, citrate salt, formate salt, trifluoro acetate salt, or trichloro acetate salt, most preferably oxalate salt.

### Step (e):

In some embodiment of the invention, filtering the precipitated salt of step (d) may be done as known in the state of art. In one preferred embodiment the precipitated salt of step (d) is filtered through cleaned nutsche filter on 5 microns cloth.

### Steps (f) and (g):

In some embodiment of the invention, the precipitated salt after filtering as mentioned in the step (e) is washed with fresh and purified polar aprotic solvent to remove the impurities. The impurities as mentioned are mostly coloured impurities such as chlorophyll or other plant matters, oils, resins, or wax.

The polar aprotic solvent employed in step (f) for washing the obtained precipitated salt may be selected from a group comprising of acetone, acetonitrile, dimethyl sulfoxide, dimethylformamide, tetrahydrofuran, dichloromethane, ethyl acetate, or mixture thereof, preferably the solvent is acetone.

This step of washing with organic solvent may be performed multiple times till the desired colour as light greenish colour to light yellowish colour is obtained.

### Step (h):

The salt product obtained after washing is dried by the processes as known in the state of art. Traditionally known methods for drying are sun drying, air drying, contact drying, infrared drying, freeze-drying, fluidized bed drying, dielectric drying etc.

In one preferred embodiment, the salt product obtained after washing in Step (f) and/or (g) of the present process is air dried for 5-6 hours and then loaded into Vacuum Tray Dryer at 45-50° C. under 700 mm of Hg to obtain high purity Mitragynine salt.

The purified Mitragynine salt obtained in step (h) of the present process is purified Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt, most preferably purified Mitragynine oxalate salt.

In some embodiment of the invention there is provided a process for the purification of Mitragynine from the crude extract comprising the steps of:
(a) mixing crude extract of Mitragynine with fresh and purified acetone in the reactor followed by stirring for 2 hours at 30-50 RPM;
(b) filtering the stirred solution as obtained in step (a) to obtain filtered solution and spent;
(c) again adding fresh and purified acetone to the spent obtained in step (b), followed by stirring for 2 hours at 30-50 RPM and filtering, to obtain filtered solution;
(d) mixing the filtered solution obtained in step (b) and step (c) and oxalic acid solution in the reactor followed by stirring at 30-50 RPM for 2 hours until Mitragynine oxalate salt precipitate is formed;
(e) filtering the precipitated Mitragynine oxalate salt as obtained in step (d);
(f) washing the filtered precipitated Mitragynine oxalate salt of step (e) with fresh and purified acetone to obtain the Mitragynine oxalate salt product;
(g) optionally performing step (f) again to obtain the Mitragynine oxalate salt product; and
(h) drying the salt product obtained in step (f) and/or step (g) to form purified Mitragynine oxalate salt.

In some embodiment of the invention, the oxalic acid solution as used in the step (d) of the present invention is prepared by dissolving oxalic acid in acetone with continuous mixing followed by stirring for 30 minutes-2 hours, preferably 1 hour at 10-50 RPM, followed by filtering. Filtering may be done as known in the state of art or as described above. In one preferred embodiment filtration is carried through 100 mesh cloth.

In one preferred embodiment, the Mitragynine oxalate salt product obtained after step (f) and/or step (g) of the present process is air dried for 5-6 hours and then loaded into Vacuum Tray Dryer at 45-50° C. under 700 mm of Hg, to obtain high purity Mitragynine oxalate salt.

The obtained Mitragynine oxalate salt in the present process is in amorphous form.

The process of the present invention provides improved yield of Mitragynine oxalate salt (32%) with purity of 62-68% by HPLC.

In some embodiment of the invention, highly pure Mitragynine oxalate salt obtained according to present invention is suitable for the pharmaceutical preparations.

Certain specific aspect and embodiment of the present invention will be explained in detail with reference to the following examples, which are provided only for purposes of illustration and should not be construed as limiting the scope of the invention in any manner.

### EXAMPLES

### Crude Mitragynine Extract:

Crude Mitragynine extract can be obtained by traditionally known methods such as maceration, percolation, reflux, Solvent Extraction, Soxhlet extraction etc. For the present process, the crude extract of Mitragynine was obtained by the novel process for extraction of Mitragynine from Mitragyna speciosa plant, which is developed by the present inventor and is disclosed and filed separately in another Indian patent application No. IN 202341010687 filed in the Indian Patent Office on Feb. 17, 2023.

### Example 1: Dissolution of Mitragynine from Crude Extract in Acetone

A fresh and purified acetone (720 Liters) was added to the reactor of volume capacity 1000 L. The crude Mitragynine extract (60 Kgs) was slowly added to the reactor and mixed with acetone. The mixture was kept under stirring by agitator at 30-50 RPM, at room temperature for 2 hours. After stirring, the obtained solution was filtered into a cleaned nutsche filter with volume capacity from 1000 L on 5-micron cloth to separate filtered solution and spent. Fresh and purified acetone (60 litres) was again added to the spent followed by stirring for 2 hours at 30-50 RPM and filtering through cleaned nutsche filter on 5 microns cloth to obtain filtered solution.

The filtered solution obtained above was combined and loaded into the cleaned reactor.

### Example 2: Preparation of Precipitated Mitragynine Oxalate Salt

Oxalic acid (12 Kgs) was added slowly to fresh and purified acetone (80 litres) with continuous mixing to allow oxalic acid to dissolve in acetone completely. This is followed by stirring for 1 hour at 30-50 RPM and filtering through 100 mesh cloth to obtain oxalic acid solution.

The oxalic acid solution as obtained above was added to the filtered solution loaded into the cleaned reactor. The obtained solution was stirred for 2 hours at 30-50 RPM, until the Mitragynine oxalate salt precipitate is formed. The precipitate salt was filtered through cleaned nutsche filter on 5 microns cloth.

### Example 3: Washing Precipitated Mitragynine Oxalate Salt with Acetone

The precipitated salt obtained in Example-2 was loaded to the reactor. The loaded salt in the reactor was washed with 180 L to 200 L of fresh and purified acetone. After washing with an acetone, the solution was checked for the desired colour such as light greenish to light yellow.

If the desired colour was not achieved, the precipitated salt was again washed with 50 L to 100 L of fresh and purified acetone and check for the desired colour.

The obtained salt product was of light greenish to light yellowish colour.

### Example 4: Drying Mitragynine Oxalate Salt Product

The obtained salt product was subjected to air drying for 5 to 6 hours and then it was loaded into Vacuum Tray Dryer 45-50° C. under 700 mm of Hg to obtain purified amorphous solid powder of Mitragynine oxalate salt which was Light greenish to light yellowish colour.

All the steps are performed at room temperature.

Yield: 32%

### Example 5: Purity of Obtained Salt by HPLC

The above process has been performed with 3 repetitions.

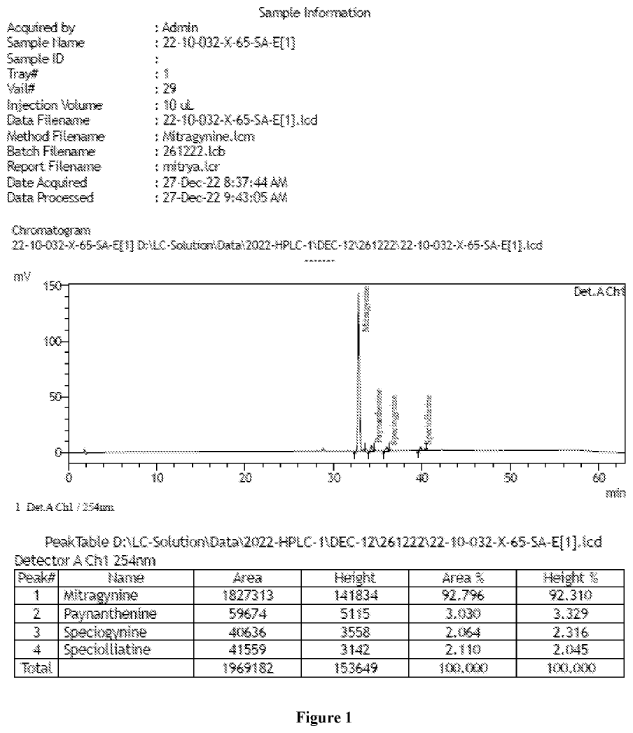

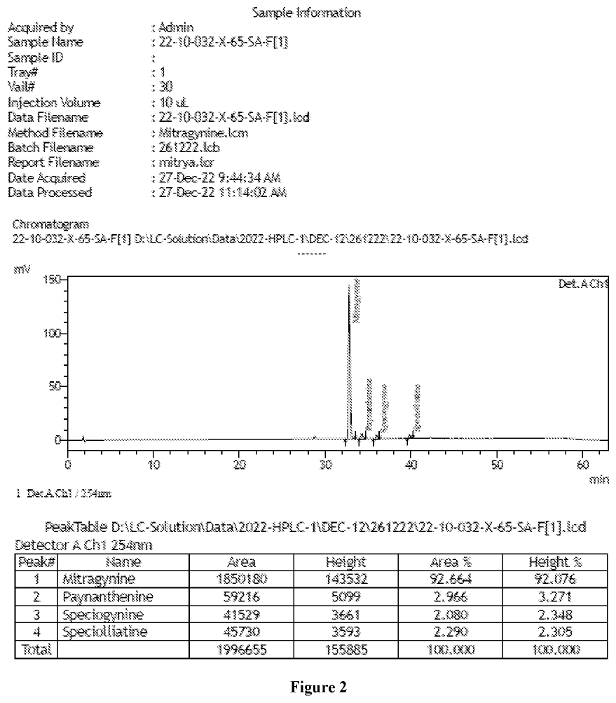

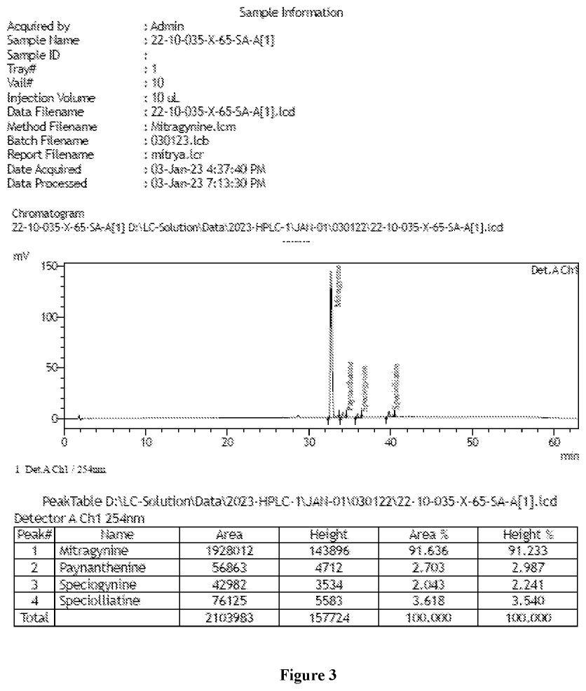

The obtained purified amorphous solid powder of Mitragynine oxalate salt from 3 repetitions were analysed through HPLC for purity (FIGS. 1-3).

Purity: 65% by HPLC.

Other impurities: Paynanthenine, Speciogynine, and Speciolliatine.

What is claimed is:

1. A process for purification of Mitragynine from crude extract of Mitragynine comprising the steps of:
(a) mixing crude extract of Mitragynine with polar aprotic solvent in a reactor followed by stirring;
(b) filtering the stirred solution obtained in step (a) to obtain filtered solution and spent;
(c) optionally, adding polar aprotic solvent to the spent obtained in step (b), followed by stirring and filtering, to obtain filtered solution;
(d) mixing the filtered solution obtained in step (b) and/or step (c) and an acid solution in the reactor followed by stirring until Mitragynine salt precipitate is formed;
(e) filtering the precipitated salt as obtained in step (d);
(f) washing the filtered precipitated salt of step (e) with polar aprotic solvent to obtain the salt product;
(g) optionally, performing step (f) multiple times to obtain the salt product; and
(h) drying the salt product obtained in step (f) and/or step (g) to form purified Mitragynine salt.

2. The process as claimed in claim 1, wherein the polar aprotic solvent employed in steps (a) and (c) is selected from a group comprising of acetone, acetonitrile, dimethyl sulfoxide, dimethylformamide, tetrahydrofuran, dichloromethane, ethyl acetate, or mixture thereof.

3. The process as claimed in claim 1, wherein the polar aprotic solvent employed in steps (a) and (c) is acetone.

4. The process as claimed in claim 1, wherein in step (a) the stirring is carried out for 2 hours at 30-50 RPM.

5. The process as claimed in claim 1, wherein in step (b) filtering is carried out through cleaned nutsche filter on 5 microns cloth.

6. The process as claimed in claim 1, wherein the acid solution in step (d) is an organic or mineral acid solution.

7. The process as claimed in claim 6, wherein the organic or mineral acid solution is prepared by dissolving the organic or mineral acid slowly into a solvent with continuous mixing followed by stirring at 30-50 RPM for about 1 hour and filtering.

8. The process as claimed in claim 7, wherein the organic or mineral acid is selected from a group comprising of hydrochloric acid, hydrobromic acid, perchloric acid, sulphuric acid, oxalic acid, tartaric acid, citric acid, formic acid, trifluoro acetic acid, trichloro acetic acid, or mixture thereof.

9. The process as claimed in claim 8, wherein the organic acid is oxalic acid.

10. The process as claimed in claim 7, wherein the solvent is organic solvent selected from a group comprising of chloroform, acetone, methanol, ethanol, n-butanol, isopropyl alcohol, ethyl acetate, diethyl ether, n-hexane, methylene dichloride, ethylene dichloride or mixture thereof.

11. The process as claimed in claim 10, wherein the solvent is acetone.

12. The process as claimed in claim 7, wherein filtering is carried out through 100 mesh cloth.

13. The process as claimed in claim 1, wherein the organic acid solution is oxalic acid solution.

14. The process as claimed in claim 1, wherein in step (d) the stirring is carried out for about 2 hours at 30-50 RPM.

15. The process as claimed in claim 1, wherein the Mitragynine salt precipitate formed in step (d) is hydrochloride salt, hydrobromide salt, perchlorate salt, sulphate salt, oxalate salt, tartrate salt, citrate salt, formate salt, trifluoro acetate salt, or trichloro acetate salt.

16. The process as claimed in claim 1, wherein in step (d) the Mitragynine salt precipitate is Mitragynine oxalate salt precipitate.

17. The process as claimed in claim 1, wherein the polar aprotic solvent employed in step (f) is selected from a group comprising of acetone, acetonitrile, dimethyl sulfoxide, dimethylformamide, tetrahydrofuran, dichloromethane, ethyl acetate, or mixture thereof.

18. The process as claimed in claim 1, wherein the polar aprotic solvent employed in step (f) is acetone.

19. The process as claimed in claim 1, wherein in step (h) the salt product is air dried for 5-6 hours followed by drying in Vacuum Tray Dryer at 45-50° C. under 700 mm of Hg.

20. The process as claimed in claim 1, wherein the purified Mitragynine salt obtained in step (h) is purified Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt.

21. The process as claimed in claim 1, wherein in step (h) the purified Mitragynine salt is purified Mitragynine oxalate salt.

22. The process as claimed in claim 1, wherein the obtained Mitragynine oxalate salt is in amorphous form.

23. The process as claimed in claim 1, wherein the purity of obtained Mitragynine oxalate salt is 62-68% by HPLC.

24. The process as claimed in claim 1, wherein said crude Mitragynine extract is obtained from Mitragyna speciosa.

25. A process for purification of Mitragynine from crude extract of Mitragynine comprising the steps of:
(a) mixing crude extract of Mitragynine with fresh and purified acetone in the reactor followed by stirring for 2 hours at 30-50 RPM;
(b) filtering the stirred solution as obtained in step (a) to obtain filtered solution and spent;
(c) adding fresh and purified acetone to the spent obtained in step (b), followed by stirring for 2 hours at 30-50 RPM and filtering, to obtain filtered solution;
(d) mixing the filtered solution obtained in step (b) and step (c) and oxalic acid solution in the reactor followed by stirring at 30-50 RPM for 2 hours until Mitragynine oxalate salt precipitate is formed;
(e) filtering the precipitated Mitragynine oxalate salt as obtained in step (d);
(f) washing the filtered precipitated Mitragynine oxalate salt of step (e) with fresh and purified acetone to obtain the Mitragynine oxalate salt product;
(g) optionally performing step (f) again to obtain the Mitragynine oxalate salt product; and
(h) air drying the salt product obtained in step (f) and/or step (g) for 5-6 hours followed by drying in Vacuum Tray Dryer at 45-50° C. under 700 mm of Hg to form purified Mitragynine oxalate salt.

26. The process as claimed in claim 25, wherein the oxalic acid solution of step (d) is prepared by dissolving oxalic acid in acetone with continuous mixing followed by stirring for 1 hour at 10-50 RPM and filtering through 100 mesh cloth.

27. The process as claimed in claim 25, wherein the purity of obtained Mitragynine oxalate salt is 62-68% by HPLC.

28. The process as claimed in claim 25, wherein said crude Mitragynine extract is obtained from Mitragyna speciosa.

29. Mitragynine oxalate salt having 62-68% HPLC purity.
